(19) United States
(12) Patent Application Publication
(10) Pub. No.: US 2017/0160200 A1
(43) Pub. Date: Jun. 8, 2017

(54) OPTICAL ANALYSIS DEVICE

(71) Applicant: HITACHI, LTD., Chiyoda-ku, Tokyo (JP)

(72) Inventors: Hideharu MIKAMI, Tokyo (JP); Masataka SHIRAI, Tokyo (JP)

(73) Assignee: HITACHI, LTD., Chiyoda-ku, Tokyo (JP)

(21) Appl. No.: 15/308,656

(22) PCT Filed: May 26, 2014
(86) PCT No.: PCT/JP2014/063852

§ 371 Date: Nov. 3, 2016

(51) Int. Cl. G01N 21/65; C12Q 1/68; G01N 33/53; G01N 15/14

(52) U.S. Cl. CPC G01N 21/65; G01N 15/1468; C12Q 1/68; G01N 33/5302; G01N 2021/653; G01N 2201/06113; G01N 2201/0697; G01N 2015/0065

(57) ABSTRACT

Spectral data such as a CARS spectrum of a sample is acquired at high speed by reducing the amount of data. During scan by emission light focused and emitted onto the sample, the exposed state of a detection unit of a spectroscope that divides light generated from the sample is continued, thereby acquiring spectral data obtained by summing spectra generated at a plurality of positions in the sample.

### TECHNICAL FIELD

The present invention relates to a higher-performance optical analyzing apparatus.

### BACKGROUND ART

Optical microscopes are, needless to say, observation tools that are indispensable in the field of natural science, engineering, and industries. Especially, a more sophisticated microscope including a laser as an illumination light source has been recently essential for the development of advanced technology. A typical example of such a microscope is a fluorescence confocal microscope, which is widely used in combination with a fluorescent reagent in the field of medicine and biology as means to observe the distribution of a specific substance in a biological sample. Coupled with a high-performance short-pulse laser light source becoming available in recent years, techniques for a non-linear optical microscope based on non-linear optical effects have been developed, and needs therefor in the field of medicine and biology have been grown noticeably. Known examples of such a non-linear optical microscope (or non-linear microscope) include a two-photon fluorescence microscope (Nonpatent Literature 1), an SHG microscope (Nonpatent Literature 2), a coherent anti-stokes Raman scattering (CARS) microscope (Nonpatent Literature 3), and a stimulated Raman scattering (SRS) microscope (Nonpatent Literature 4). For instance, a two-photon fluorescence microscope allows a small wavelength band less absorbing a sample to be selected as laser light to be applied to the sample, and so imaging is enabled at a deep part as compared with a conventional fluorescence confocal microscope. An SHG microscope is to observe the second harmonics from a sample, which can detect the fiber structure of collagen or the like and a specific structure such as a cell membrane selectively. A CARS microscope is configured to irradiate a sample with two types of laser lights including pump light and Stokes light, and to observe anti-Stokes light generated as a result of the resonance of the frequency difference between these lights with the natural vibration of the molecules of the sample. Based on the distribution of wavelength and intensity of the anti-Stokes light, the distribution of a specific substance in the sample can be observed, and so this technique has attracted attention as a labeling-free and non-invasive microscope as a substitute of a fluorescence microscope. A SRS microscope is configured to irradiate a sample with pump light and Stokes light similarly to the CARS microscope, and to observe the natural vibration of the substance in the form of a change in intensity of these two types of lights, which also is a non-invasive microscope like the CARS microscope. In this way, a non-linear optical microscope can provide various sophisticated observation means, which cannot be implemented with conventional microscopes.

The following describes the operating principle of the CARS microscope. CARS is the emission of light due to third-order polarization, and in order to generate CARS, pump light, Stokes light, and probe light are required. Typically, in order to reduce the light sources in number, the pump light doubles as the probe light. In this case, induced third-order polarization will be represented by:

P_{AS}⁽³⁾(ω_{AS})=|χᵣ⁽³⁾(ω_{AS})+χnr⁽³⁾|E_{P}²(ω_{P})E*_{S}(ω_{S}) (1)

In this expression, χᵣ⁽³⁾ (ω_{AS}) is the resonant term of the molecule vibrations of third-order electric susceptibility, and χₙᵣ⁽³⁾ is the non-resonant term. E_{P} represents the electric field of the pump light and the probe light, and E_{S} represents the electric field of the Stokes light. The non-resonant term does not have frequency-dependency. Asterisk attached to the shoulder of E_{S} in Equation (1) denotes a complex conjugate. Then, the intensity of CARS light is represented as follows:

I_{CARS}(ω_{AS})∝|P_{AS}⁽³⁾(ω_{AS})|² (2)

Referring to the energy level diagram illustrated in FIG. 13 of molecules, the following describes the mechanism to generate CARS light. This drawing illustrates the process for the resonant term. Numeral 1401 denotes the ground state of molecule vibrations, and numeral 1402 denotes the excitation state of vibrations. Pump light at frequency ωp and Stokes light at frequency ω_{S} are applied simultaneously. At this time, molecules are excited to some excitation level of vibrations in 1402 via a virtual intermediate state 1403. When the molecules in such an excitation state are irradiated with the probe light at frequency ω_{P}, the molecules return to the ground state of vibrations while generating CARS light at frequency ω_{AS} via a virtual intermediate state 1404. The frequency of the CARS light at this time is represented as ω_{AS}=2·ω_{P}−ω_{S}.

As is evident from FIG. 13, this resonant CARS light is generated only when the difference in frequency ω_{P}−ω_{S} between the pump light and the Stokes light is equal to a certain vibration excitation state of the sample observed. Note here that Planck units are used here, where the Planck constant is 1. That is, when a broadband light source is used for the Stokes light, the CARS light generated also becomes broadband light, and has a spectrum having a sharp peak at the wavelength corresponding to the vibration excitation state. This spectrum is called a Raman spectrum, which reflects the distribution of the vibration excitation state of the molecules in the sample, and so can be used for the identification of the molecular species.

FIG. 14 is a diagram illustrating one process related to the non-resonant term in Equation (1). The Stokes light has the frequency that is not in the vibration excitation state, and the process occurs via a virtual intermediate state 1405. When the pump light at frequency ω_{P} and probe light at frequency ω′_{P} are applied simultaneously, the virtual intermediate state 1405 involving electrons or the like is excited, and when Stokes light at frequency ω′_{S} is further applied, non-resonant CARS light at frequency ω_{AS} is generated via a virtual intermediate state 1406. This non-resonant CARS light is generated irrespective of the vibration excitation state, and so when broadband Stokes light is used, broadband non-resonant CARS light is generated, whose intensity does not have wavelength-dependency. These resonant CARS light and non-resonant CARS light are mutually coherent, and so interfere occurs therebetween. Since the spectrum of the resonant CARS light, i.e., the Raman spectrum, actually is required to identify the molecular species in the sample, signal processing has to be performed to acquire the Raman spectrum from the CARS light spectra acquired. Some methods are known for such signal processing (see Nonpatent Literature 5), and for instance, in the method of maximum entropy that is to recover a phase spectrum from the intensity spectrum, mathematical calculation is performed to find the complex component of the resonant term.

The pump light, the Stokes light, and the CARS light have a relationship for frequency as in FIG. 15. When the pump light at a predetermined frequency and the Stokes light in a frequency area smaller than that are incident on the sample, CARS light is generated in a frequency area that is larger than the pump light.

The CARS microscope is configured to measure the thus found Raman spectrum a plurality of times while changing the focusing position of the pump light and the Stokes light, and to acquire the image of the spatial distribution for each molecular species as a result.

### CITATION LIST

### Nonpatent Literature

Nonpatent Literature 1: W. Denk et al., “Two-Photon Laser Scanning Fluorescence Microscopy”, Science, Volume 248, Issue 4951, pp. 73-76 (1990)
Nonpatent Literature 2: P. J. Campagnola et al., “Second-harmonic imaging microscopy for visualizing biomolecular arrays in cells, tissues and organisms”, Nature Biotechnology 21, 1356-1360 (2003)
Nonpatent Literature 3: M. Okuno et al., “Quantitative CARS Molecular finger printing of living Cells”, Angewandte Chemie International Edition 49, 6773-6777(2010)
Nonpatent Literature 4: B. G. Saar et al., “Video-Rate Molecular Imaging in Vivo with Stimulated Raman Scattering”, Science Vol. 330 1368(2010)
Nonpatent Literature 5: J. P. R. Day, K. F. Domke, G. Rago, H. Kano, H. Hamaguchi, E. M. Vartiainen, and M. Bonn, “Quantitative Coherent Anti-Stokes Scattering (CARS) Microscopy”, J. Phys. Chem. B, Vol. 115, 7713-7725 (2011)

### SUMMARY OF INVENTION

### Technical Problem

When the sample, such as a cell, is analyzed by the CARS microscope, laser light is emitted onto the respective points in the sample so that the spectrum of CARS light is measured by the spectroscope. Then, the spectral data of the CARS light is acquired at different positions over a two- or three-dimensional region. From this data, spectral information and spatial information (image information) of the sample are acquired. However, in this case, it takes a long time to acquire the data due to the limited data transfer rate of the detection unit of the spectroscope, and so it can be difficult to measure the sample in real time. In particular, when the CARS microscope is used for an application for analyzing a large number of cells (single cell analysis), the low data acquisition speed is a fatal disadvantage. This makes it substantially difficult to apply the current CARS microscope to single cell analysis. Further, in the conventional method for acquiring spectral information and spatial information, the amount of data is enormous in itself, which makes it difficult to store measurement data and to take a long time to analyze the data. For instance, taking a long time to analyze data by using the method of maximum entropy lowers the substantial through rate of sample analysis. This is a significant problem when the CARS microscope is applied as an analyzing method. The above problems are shared between measuring methods by which spectra are acquired at the respective points in the sample (hyper spectral imaging), which is true for acquiring fluorescence spectra by a Raman microscope and a fluorescence confocal microscope, in addition to the CARS microscope.

In view of the above problems, an object of the present invention is to provide an optical analyzing apparatus that can analyze a sample at high speed.

### Solution to Problem

Hyper spectral imaging, such as the conventional CARS microscope, is based on an idea that as much information as possible (spectral information and spatial information) is acquired from a sample to facilitate analysis. However, actually, in many cases, all of acquired data is not necessary, and for instance, there may be cases where the content of a certain substance in a cell in part or in whole has only to be found. Accordingly, the present invention solves the problems by acquiring the summed value of spectra in a region in part or in whole, not by acquiring spectra from all spatial points in a sample. Specifically, the following means was used.

An optical analyzing apparatus includes a light source such as a short-pulse laser, a sample holding unit that holds a sample, an emission optical system that focuses and emits a light flux from the light source onto the sample held by the sample holding unit, a light division unit that divides light generated from the sample by light emission, a detection unit including a detector array, such as a line sensor and an area sensor, that detects the light divided by the light division unit, and an emission control unit that controls the position of light emission onto the sample by the emission optical system, in which the detection unit continues an exposed state over a plurality of positions of light emission onto the sample by the emission control unit, and outputs a spectrum obtained by summing spectra generated from the positions of light emission.

This can shorten the data acquisition time, and reduce the amount of data.

(2) In (1), the detection unit outputs a plurality of the summed spectra, and averages the plurality of outputted spectra.

This can avoid a spectroscope from being saturated even when the intensity of light measured is high. In addition, by summing and averaging the plurality of spectra, noise can be relatively reduced, thereby enabling the S/N ratio of the spectral signals to be increased.

(3) In (1), the optical analyzing apparatus further includes an image data acquisition unit that acquires image data of the sample held by the sample holding unit, and a shape recognition unit that recognizes the shape of the sample based on the acquired image data, in which the emission control unit focuses and emits a light flux from the light source onto a specific region of the sample based on the shape of the sample recognized by the shape recognition unit.

This can shorten the measurement time. In addition, spectral signals can be acquired from different portions of the sample, thereby enabling more detailed sample analysis.

(4) In (1), as the spectrum, a CARS spectrum is detected.

Thus, undyed and high-speed sample analysis is enabled.

(5) In (1), the emission control unit includes a scan mirror, and the scan mirror has a control direction perpendicular to the light division direction of the detection unit.

This makes scan faster, thereby enabling high-speed measurement.

(6) In (1), the emission control unit scans the sample in two dimensions.

This can measure a relatively thin sample at high speed.

(7) In (1), the emission control unit scans the sample in three dimensions.

This can acquire a high reliable measurement value for a relatively thick sample.

(8) A biomolecule analyzing apparatus includes a light source, a sample holding unit that holds a plurality of cells as a sample, an observation unit that observes the cells held by the sample holding unit, an emission optical system that focuses and emits a light flux from the light source onto each cell held by the sample holding unit, a light division unit that divides light generated from the cell by light emission, a detection unit that detects the light divided by the light division unit, an emission control unit that controls the position of light emission onto the cell by the emission optical system, cell destruction means that destroys the cell held by the sample holding unit, and a biomolecule capturing device that captures biomolecules in the cell released from the destroyed cell, in which the detection unit continues an exposed state over a plurality of positions of light emission onto the cell by the emission control unit, and outputs a spectrum obtained by summing spectra generated from the positions of light emission.

This can analyze the biological sample at high speed.

### Advantageous Effects of Invention

According to the present invention, it is possible to provide the high-speed optical analyzing apparatus that reduces the amount of data as compared with conventional ones.

Other problems, configurations, and effects will be apparent from the description of the following embodiments.

### BRIEF DESCRIPTION OF DRAWINGS

FIG. 1 is a schematic diagram illustrating the configuration example of an optical analyzing apparatus.

FIG. 2 is a schematic diagram of a light reception unit of a CCD camera.

FIG. 3 are sequence diagrams of data acquiring operations.

FIG. 4 is a block diagram when a scan mirror is used.

FIG. 5 is a block diagram of an optical analyzing apparatus that detects the backscattering of CARS light.

FIG. 6 is a schematic diagram illustrating the configuration example of an optical analyzing apparatus.

FIG. 7 is a schematic diagram illustrating the configuration example of an optical analyzing apparatus.

FIG. 8 is a schematic diagram illustrating the configuration example of a biomolecule analyzing apparatus.

FIG. 9 is a detailed diagram of the periphery of a sample illustrating the configuration example of a biomolecule extraction system.

FIG. 10 is a top view of a pore array sheet.

FIG. 11 is a flowchart of assistance in explaining the operation of the biomolecule analyzing apparatus.

FIG. 12 is a plot illustrating the results of principal component analysis.

FIG. 13 is an energy diagram representing a resonant CARS process.

FIG. 14 is an energy diagram representing a non-resonant CARS process.

FIG. 15 is a diagram illustrating the relationship of frequencies among pump light, Stokes light, and CARS light.

### DESCRIPTION OF EMBODIMENTS

Embodiments of the present invention will now be described with reference to the drawings.

### First Embodiment

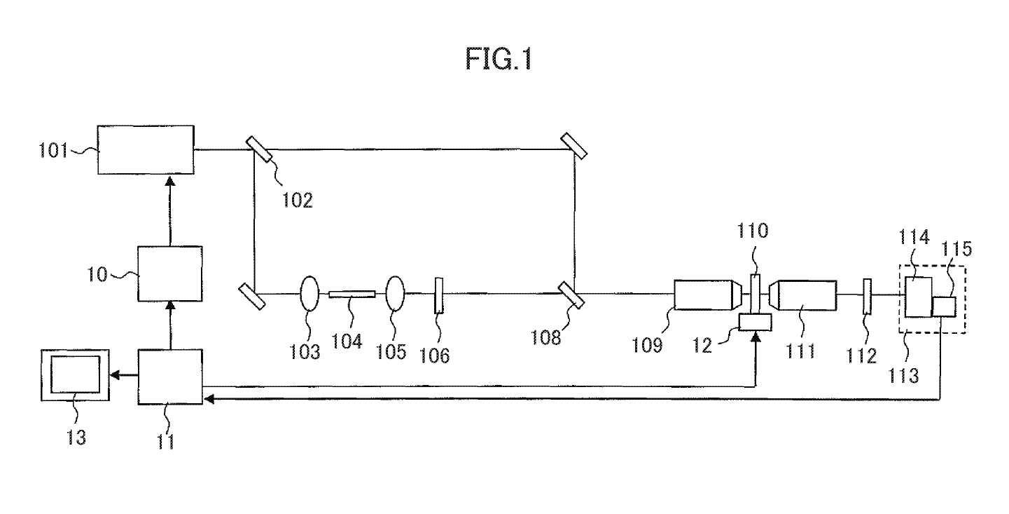

FIG. 1 is a schematic diagram illustrating the basic configuration example of an optical analyzing apparatus of the present invention. The operation in this embodiment will now be described with reference to FIG. 1.

Laser light emitted from a light source, that is, a short-pulse laser light source 101 (a center wavelength of 1064 nm, a pulse width of 900 ps, a repetition frequency of 30 kHz, an average output of 200 mW) that is controlled in light-emission by a driver 10 receiving a command from a computer 11 is divided at a beam splitter 102 into two, including transmitted light as pump light and reflected light. The reflected light is coupled with a photonic crystal fiber 104 via a focusing lens 103, whereby broadband supercontinuum light is generated inside the fiber. The thus generated supercontinuum light is made parallel light via a collimate lens 105, and is incident on a long-pass filter 106, along which components at the wavelength of the short-pulse laser light source and at the wavelengths shorter than that are blocked. Stokes light that has a component at the wavelength longer than that of the pump light and has passed through the long-pass filter 106 is multiplexed with the pump light at a dichroic mirror 108. Herein, the dichroic mirror 108 has the property of reflecting lights at the wavelength of the pump light and in the wavelength band shorter than that, and of transmitting light in the wavelength band longer than the pump light. Then, the pump light is reflected and the Stokes light is transmitted, resulting in multiplexing.

This multiplexed light flux is focused at one point in a sample 110 via an objective lens 109 (NA of 0.9, and a magnification of ×40) configuring an emission optical system that focuses and emits the light flux from the light source onto the sample, whereby CARS light is generated, which reflects the resonant vibrations of molecules present at the focusing position in the sample. The CARS light is then made parallel light via a condenser lens 111 (NA of 0.65), passes through a short-pass filter 112 that blocks the pump light and the Stokes light that are coaxial components, is incident on a spectroscope 113, is divided at a light division unit 114, and is detected by wavelength at a detection unit 115, where the spectrum is outputted as a detected signal.

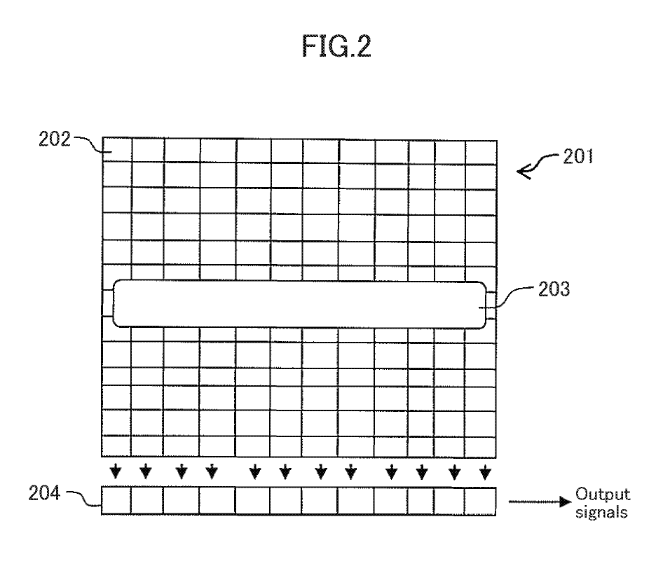

Here, the detecting operation of the spectroscope 113 will be described. The spectroscope 113 includes the light division unit 114 that diffracts incident light by wavelength in different directions by a diffraction grating, and the detection unit 115 that detects the light diffracted at the light division unit 114 by a one- or two-dimensional detector array (a CCD camera, or a CMOS camera). In this embodiment, a CCD camera is used as the detection unit 115 that has a light reception unit 201 with pixels 202 arrayed in two dimensions, as illustrated in FIG. 2. Light divided at the light division unit 114 is incident as a laterally long beam 203 on the light reception unit, and has different wavelengths according to position in the lateral direction. Here, the CCD camera as the detection unit 115 is brought into an exposed state, that is, a state in which the pixels are exposed to the incident light to convert the incident light to electric charge for accumulating the electric charge, during a predetermined time by external control. After the completion of the exposure, the total amount of the electric charge accumulated in each vertically arrayed pixel row is transferred to a buffer 204 (full vertical binning), whereby the electric charge in the buffer 204 is outputted as a serial signal to the outside. Thus, the output signal is a signal proportional to the intensity by wavelength of the incident light, that is, the spectral signal of the incident light.

Here, in this embodiment, while the detection unit 115 is in the exposed state, an XYZ stage 12 holding the sample 110 is driven so that the focusing position of the pump light and the Stokes light onto the sample scans the sample in three- or two-dimensions. More specifically, a previously designated, e.g., rectangular parallelepiped region or rectangular region is scanned at a constant speed. Thus, one type of spectral signal is acquired in one sample measurement. The one type of spectrum corresponds to a spectrum obtained by summing spectra generated from the respective positions in the sample on the scan line. The number of data pieces is the number of pixels in the lateral direction of the CCD camera. The acquired signal will be hereinafter called a CARS spectrum. In the conventional method, a large number of CARS spectra are acquired as data because they are acquired each time the focusing position of the pump light and the Stokes light is changed.

The CARS spectra acquired in this embodiment are subjected to signal processing, such as the method of maximum entropy, to be converted to Raman spectra. The Raman spectra acquired here represent the contents of various chemical species in the sample. The CARS spectra acquired in this embodiment are signals acquired by scanning the position of the pump light and the Stokes light. Thus, from these signals, the total contents of the chemical species in the entire sample (in the scan region) can be found.

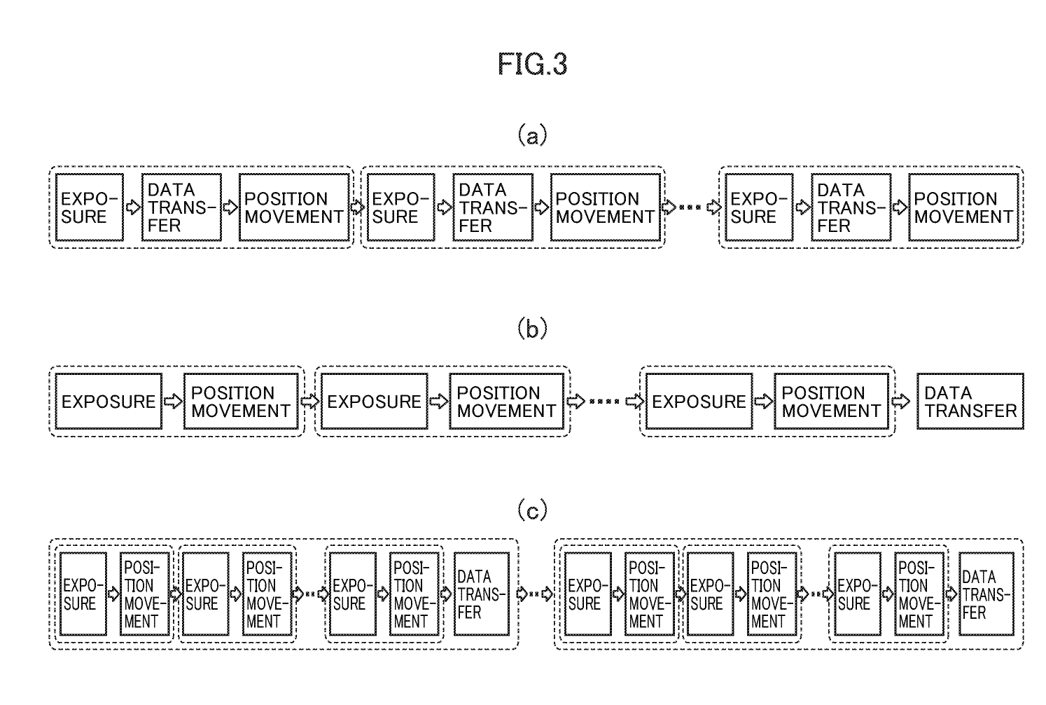

The data acquisition sequence according to this embodiment will be described with reference to FIGS. 3(a) to 3(c). FIG. 3(a) represents the sequence of the conventional method, which repeats an operation including exposure, data transfer, and position movement by the number of data. The data transfer and the position movement may be carried out in reverse order or simultaneously. FIG. 3(b) illustrates the sequence in this embodiment, which repeats an operation including the exposure and the position movement until the scan for the sample is completed, and finally carries out the data transfer. In FIG. 3(b), the exposure, position movement, and data transfer are serially carried out, but the exposure immediately before the position movement may be continued during the position movement, or the data transfer may be carried out simultaneously with the position movement immediately before the data transfer.

This embodiment and the conventional method are compared for the data acquisition time and the amount of data. The data acquisition time in the conventional method is obtained by multiplying the sum of the exposure time, the movement time, and the spectral data transfer time of one spectral measurement by the number of measurement points (the number of measuring positions on the sample space). On the contrary, the data acquisition time in this embodiment is approximately the data acquisition time in the conventional method when the data transfer time is assumed to be 0. Thus, when the exposure time is equal to or shorter than the data transfer time, the data acquisition time can be shortened. The amount of data in the conventional method is obtained by multiplying the amount of data in this embodiment by the number of measurement points. Typically, the number of measurement points is some tens of thousands to some millions to acquire an image. Thus, by this embodiment, the amount of data is reduced to the order of a fraction of some millions to a fraction of some tens of thousands.

The sample position scan in this embodiment may fix the position of the sample during the exposure discretely, that is, at each measurement point, thereby moving it to a different position after the completion of the exposure, or may change the position of the sample continuously, that is, at a predetermined speed. The continuous scan continues to scan the light spot in the sample during the exposure time of the detection unit, and then ends the exposure of the detection unit at the completion of the scan, thereby carrying out the data transfer. The continuous scan can be equivalent to the discrete scan because one measurement point in the conventional method corresponds to the spatial region of the focusing spot size of the pump light and the Stokes light in the sample. That is, the continuous scan is almost equal to the discrete scan when the amount of the position movement is the focusing spot size and the exposure time per measurement point is a pixel dwell time. The pixel dwell time is defined as (the focusing spot size)/(the speed at which the sample is scanned).

In this embodiment, an emission control unit that controls the position of light emission onto the sample by the emission optical system uses the XYZ stage 12 to scan the position of the sample for scanning the measurement point, but the method for controlling the position of light emission by the emission control unit is not limited to this. For instance, as the emission control unit, a scan mirror such as a galvano mirror or a MEMS mirror that scans the incidence angle of the pump light and the Stokes light onto the sample by external control may be used, or the position of the objective lens 109 may be scanned. Alternatively, a combination of the above methods may be used.

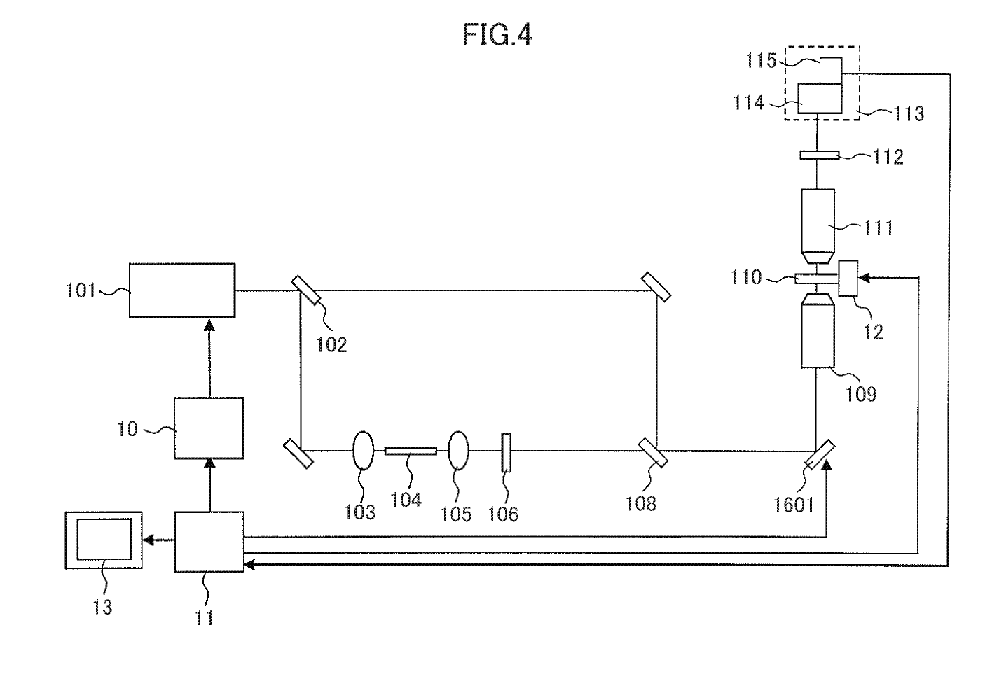

In particular, an example in which a galvano mirror is used to scan one axis will be described with reference to FIG. 4. In this case, a galvano mirror 1601 is inserted between the dichroic mirror 108 and the objective lens 109, so that pump light and Stokes light are reflected to be incident on the objective lens 109. Here, the disposition angle of the galvano mirror 1601 is controlled by external control from the computer 11, so that the angle of the light flux of the pump light and the Stokes light can be controlled. The pump light and the Stokes light whose angle is changed by the galvano mirror 1601 are focused to a position in the sample 110 different from a position before the angle is changed, and generated CARS light is incident to a different position in the light reception surface of the CCD camera. Here, the angle scan direction of the galvano mirror 1601 is set so that the position of the CARS light is changed in the perpendicular direction in FIG. 2 (the direction almost perpendicular to the light division direction) in the light reception surface of the CCD camera. In this case, the beam 203 of the CARS light travels in the perpendicular direction, but since as described above, data summed in the perpendicular direction are outputted at the time of data acquisition, output signals are not affected even when the position of the beam is changed. Other axes are scanned by using the XYZ stage 12. This operation is the same as the case of using other scan mirrors, such as a MEMS mirror. These scan mirrors are typically operated faster than the XYZ stage, so that the application of these enables high-speed measurement.

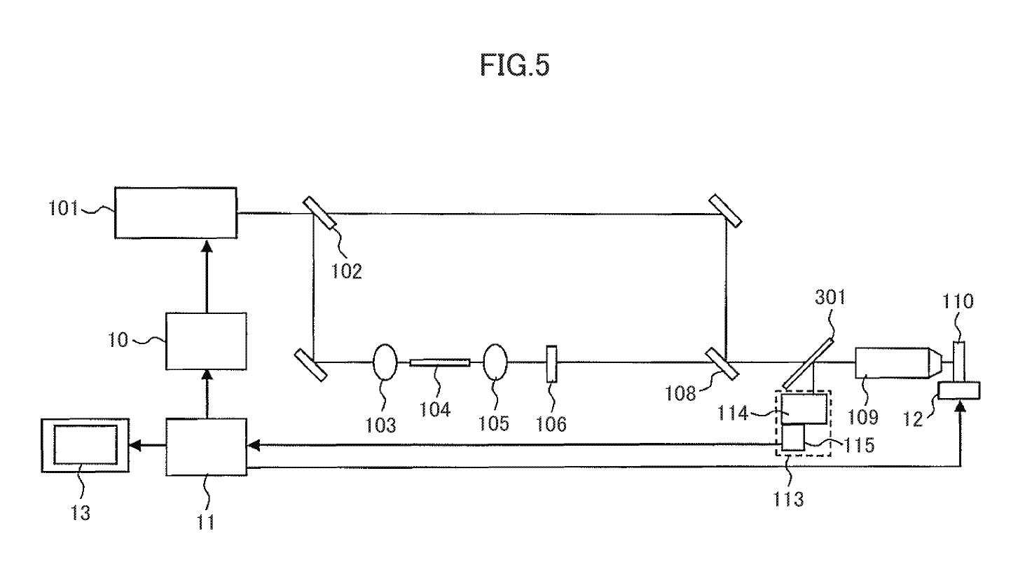

In addition, in this embodiment, the spectroscope is disposed on the opposite side of the incident side of the pump light and the Stokes light onto the sample, but may be disposed on the same side so that backscattering light from the sample is made parallel light at the objective lens 109 to be detected by the spectroscope. In this case, as illustrated in the schematic diagram in FIG. 5, the pump light, the Stokes light, and the CARS light are coaxial. Consequently, the CARS light is required to be split from the pump light and the Stokes light by using a beam splitter 301.

In this embodiment, as the detector, the CCD camera is assumed, but the detector is not limited to this, and the same effect can be obtained even when a CMOS camera or a line sensor as a one-dimensional detector array is used.

The scan in this embodiment may be carried out in two- or three-dimensions, but for a relatively thick sample (roughly, above the focal depth of the pump light and the Stokes light focused onto the sample), the three-dimensional scan is used so that the sum amount of signals from the entire sample can be precisely acquired, which is effective. On the contrary, for a thin sample (below the focal depth of the pump light and the Stokes light focused onto the sample), the two-dimensional scan is carried out so that the sum amount of signals can be precisely acquired for a short time.

### Second Embodiment

In this embodiment, the exposing operation is carried out a plurality of times for measuring the sample. The configuration example of an optical analyzing apparatus in this embodiment is the same as the first embodiment.

FIG. 3(c) illustrates a data acquisition time sequence in this embodiment. Its basic method is equal to the first embodiment, but in this embodiment, repeated is an operation in which the exposed state of the detection unit 115 is not continued throughout the entire scan for the sample, the exposed state of the detection unit 115 is stopped in the middle to carry out the data transfer, and the detection unit 115 is brought into the exposed state again. After the completion of the data acquisition, the average value of a plurality of acquired spectral data pieces is used as finally acquired data to carry out the signal processing like the first embodiment. That is, in this embodiment, the scan that is carried out throughout the entire desired region of the sample by the pump light and the Stokes light is divided into a plurality of scans, and the detection unit 115 of the spectroscope 113 then outputs a summed spectrum, like the first embodiment, during each of the divided partial scans. Thus, summed spectra equal in number to that of the partial scans are acquired, and are then averaged to be final spectral data.

In this case, one exposure time is shorter than the first embodiment, so that it is possible to avoid the saturation of the light reception unit causing failed normal data output. In addition, a plurality of data pieces are averaged to average noises added for respective spectral data outputs (caused mainly in an amplifier that converts electric charge to voltage), so that the S/N ratio can be higher than the first embodiment.

Needless to say, one exposure of the detection unit in this embodiment is required to be carried out over a plurality of positions in the sample. In other words, the exposure time of the detection unit is required to be longer than the pixel dwell time. In the conventional method, the exposure time and the pixel dwell time are equal.

### Third Embodiment

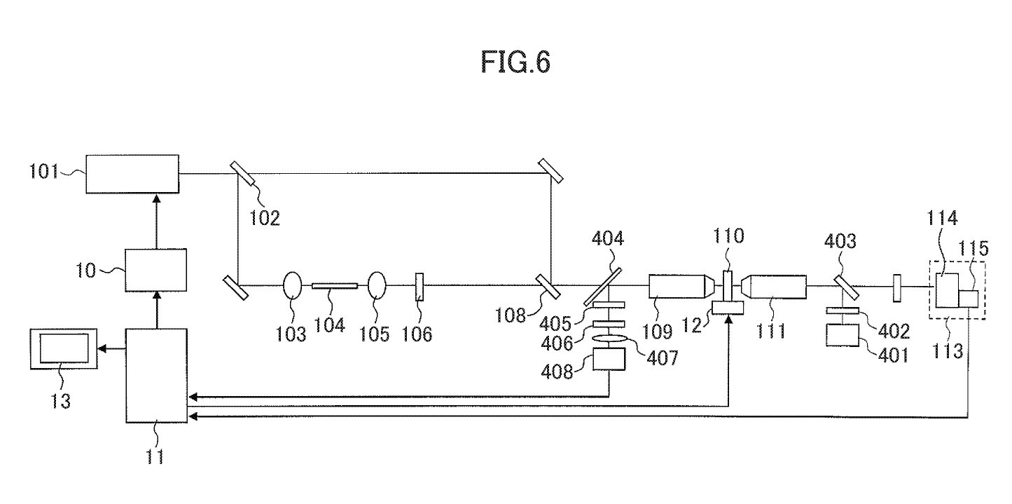

In this embodiment, data is acquired from a specific region of the sample. FIG. 6 is a schematic diagram illustrating the configuration example of an optical analyzing apparatus in this embodiment. The optical analyzing apparatus in this embodiment includes, in addition to the configuration of the optical analyzing apparatus in the first embodiment, a configuration capable of observing the sample by a differential interference microscope.

In this embodiment, illumination light from a light 401 (halogen lamp) is passed through a Wollaston prism 402, is reflected at a dichroic mirror 403, and is focused onto the sample 110 at the condenser lens 111, so that the differential interference image of the sample 110 is image-formed onto an imaging device, such as a CCD camera 408, by using the objective lens 109, a dichroic mirror 404, a Wollaston prism 405, a polarizer 406, and an image forming lens 407, thereby acquiring the image of the sample. This configuration is the same as the configuration of a well-known differential interference microscope. The dichroic mirrors 403, 404 are designed to reflect the wavelength of the visible light range of the light 401 (400 nm to 700 nm) and to transmit pump light, Stokes light, and CARS light (all of them have a wavelength in a near-infrared range above 700 nm), and do not affect CARS signal generation and detection.

Here, the image acquired at the CCD camera 408 is transmitted to the computer 11. Then, the computer 11 analyzes the image data for extracting the contour of the sample (such as a cell) at a shape recognition unit that recognizes the shape and structure of the sample. The computer 11 transmits, to the stage 12, a command to scan only the inside of the range of the contour. During the scan time, the detection unit 115 of the spectroscope 113 continues the exposed state to acquire a summed CARS spectrum. At this time, since the scan range of the light spot is limited to the sample measured, the data acquisition time can be shorter than the first embodiment. In addition, the scan range is not always the entire sample, and a CARS spectrum can be acquired from one region, e.g., from only the nuclear portion of the cell. Also in this case, a differential interference image is acquired to extract the contour of the nuclear portion by the computer 11 to scan only the nuclear portion. Further, a CARS spectrum may be acquired from each of, e.g., a plurality of locations in the same sample (e.g., the nuclear of the cell and other portions).

In this embodiment, the differential interference microscope is used as means for observing the sample. However, since the image data that can extract the contour of the sample has only to be acquired, the differential interference microscope may be replaced with an image data acquisition unit such as a typical bright-field microscope (equivalent to a configuration except for the Wollaston prisms 402, 405, and the polarizer 406) and a phase contrast microscope, or a combination of these may be used.

### Fourth Embodiment

In this embodiment, a spontaneous Raman spectrum and a fluorescence spectrum are acquired in place of a CARS spectrum.

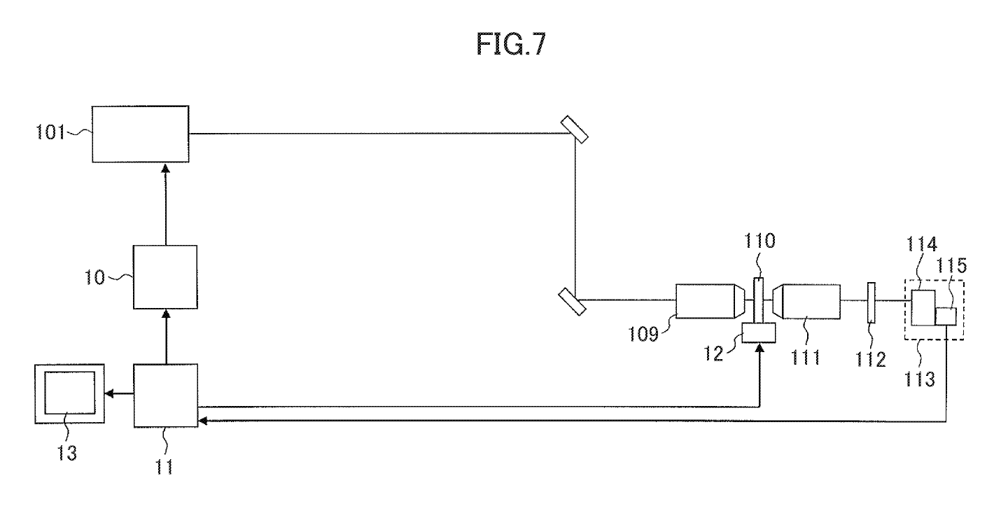

FIG. 7 is a schematic diagram illustrating the configuration example of an optical analyzing apparatus in this embodiment. In the optical analyzing apparatus in this embodiment, the Stokes light generation unit is removed from the optical analyzing apparatus illustrated in the first embodiment. That is, pump light emitted from the laser 101 is directly incident on the objective lens 109. In addition, the spectrum acquisition range in the spectroscope 113 is set to the long wavelength side from the pump light unlike CARS. This setting is carried out by setting the angle of the diffraction grating provided in the light division unit 114 in the spectroscope 113.

The operation in this embodiment is the same as the first and second embodiments, and a spectrum that reflects the content of chemical species in the sample or the fluorescence label is acquired according to the sequence illustrated in FIG. 3(b) or 3(c). To acquire a spontaneous Raman spectrum and a fluorescence spectrum from a focused laser, in the conventional method, spectral data is acquired at each focusing location for analyzing the entire sample. However, by this embodiment, the data acquisition speed can be higher and the amount of data can be reduced.

### Fifth Embodiment

In this embodiment, a biomolecule analyzing apparatus in which the optical analyzing apparatus of the present invention is applied to single cell analysis, and a CARS spectrum is acquired as one form of cell analysis.

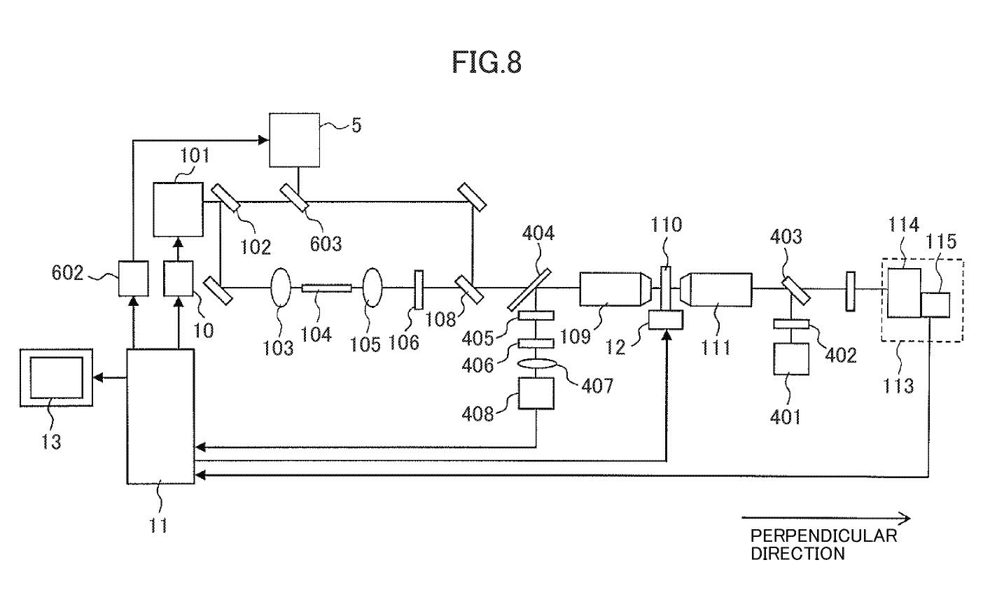

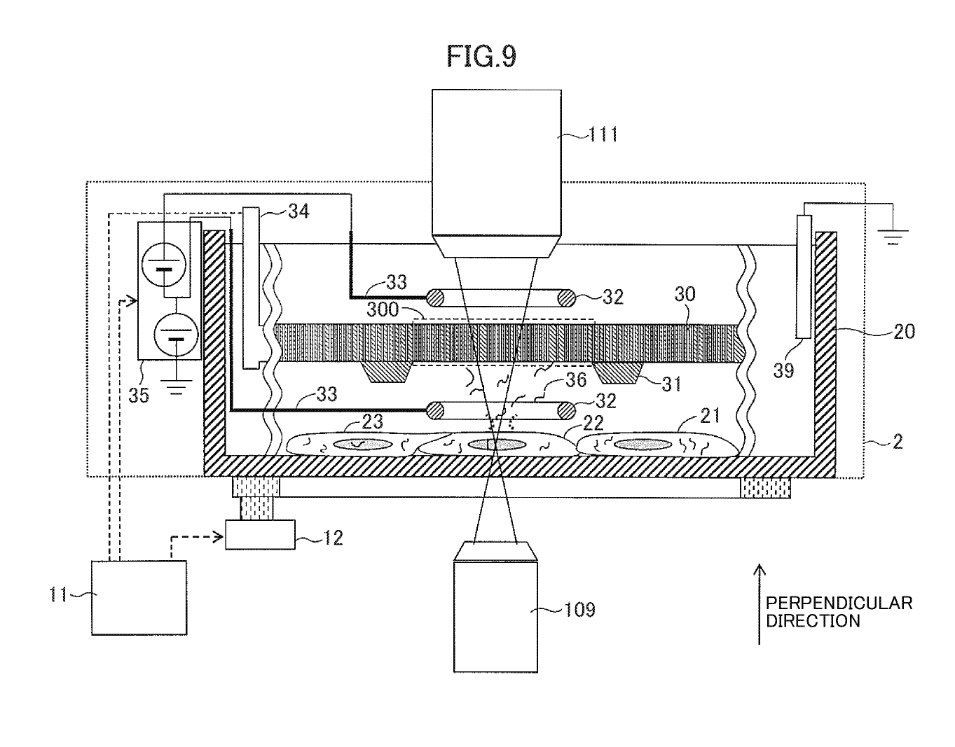

FIGS. 8 and 9 are schematic diagrams illustrating the configuration example of the biomolecule analyzing apparatus according to this embodiment. FIG. 8 is a schematic diagram illustrating the optical system portion of this apparatus, and FIG. 9 is a detailed diagram of the periphery of a sample illustrating the configuration example of a biomolecule extraction system. FIG. 9 includes a biomolecule extraction system 2 that captures mRNAs in a cell as a sample to analyze gene expression. The optical system portion and the biomolecule extraction system are controlled by the computer 11 to acquire data.

### (The Description of the Optical System Portion)

The optical system portion of the apparatus illustrated in FIG. 8 includes, in addition to the configuration illustrated in FIG. 6 in the third embodiment, a cell destruction laser 5 (a pulse laser having a wavelength of 355 nm, an average output of 2 W, and a repetition frequency of 5 kHz), a driver 602, and a dichroic mirror 603 for allowing light emitted from the laser 5 to be coaxial with pump light. The optical system portion includes three functions: (1) acquiring a differential interference microscope image, (2) acquiring a CARS spectrum, and (3) destroying a cell. The functions (1) and (2) are as described in the third embodiment. The function (3) focuses the light emitted from the cell destruction laser 5 onto a cell to be observed by the objective lens 109, and destroys the cell to release biomolecules, such as mRNAs, therein, to the outside. The released mRNAs are captured and analyzed by the biomolecule extraction system 2, as described later.

### (The Description of the Biomolecule Extraction System)

The biomolecule extraction system 2 illustrated in FIG. 9 includes an array device in which a plurality of regions for capturing biomolecules such as mRNAs released from cells are arrayed. For instance, mRNAs in each cell are captured into each region in the array device, and a reverse transcription reaction is then carried out in the array device to construct a cDNA library. In this embodiment, the array device is constructed of a transparent porous membrane in which a large number of through-holes are formed to be perpendicular to its surface, and will be hereinafter called a pore array sheet 30. In addition, the pore array sheet 30 formed with the cDNA library is called a cDNA library pore array sheet.

In this embodiment, as the pore array sheet 30, used is a porous membrane of an aluminum oxide that has a thickness of 80 μm, and a size of 2 mm×2 mm, and in which a large number of through-holes having a diameter of 0.2 μm are formed by anodic oxidation. In the pore array sheet 30, isolation walls 31 can be formed to isolate the regions for capturing biomolecules. The isolation walls 31 can be formed of, e.g., polydimethylsiloxane (PDMS) by a semiconductor process so as to have a thickness of approximately 80 μm, and can be brought into contact with the pore array sheet 30.

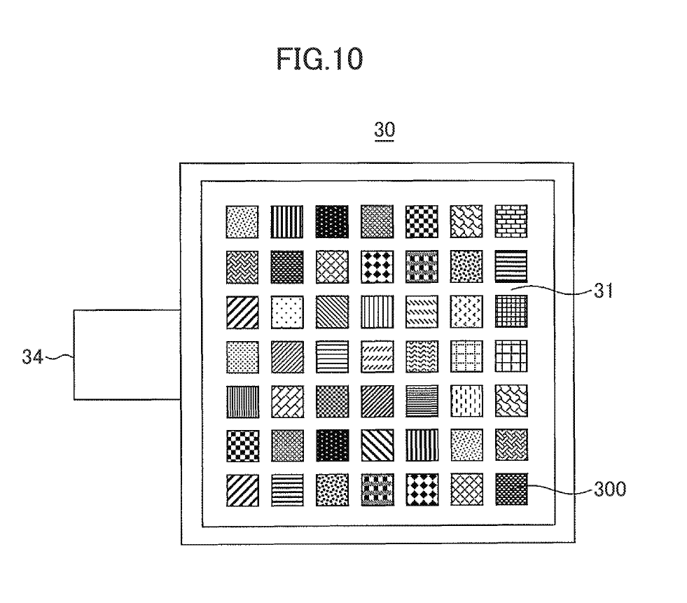

FIG. 10 is a top view of the pore array sheet 30. In the pore array sheet 30 (a size of 2 mm×2 mm, a thickness of 80 μm), a large number of regions 300 for capturing biomolecules, e.g., mRNAs, are formed. Here, each region 300 has one side of 100 μm, and the interval between it and the adjacent region of 80 μm (disposed at the pattern of 180 μm). The size of the region 300 can be freely designed to the order of 1 μm to 10 mm according to the amount of biomolecules to be captured and easiness of diffusion in its plane (the size of the molecules).

As the array device, in addition to the pore array sheet 30 made of the porous membrane formed by anodically oxidizing aluminum, a large number of through-holes may be formed by anodically oxidizing a silicon material. Further, the array device may be constructed by providing a large number of through-holes in a thin film of a silicon oxide or a silicon nitride by using a semiconductor process.

As illustrated in FIG. 9, as means for guiding biomolecules released from a cell into a specific region of the pore array sheet 30 by electrophoresis, a looped platinum electrode 32 is joined to the end of a shield wire 33. The wire of the platinum electrode 32 has a diameter of 30 μm, and is folded into two to twist its lead wire joining portions to form one wire. The loop side is then processed to have a circular shape having a diameter of 100 μm. Two such electrodes are made, and are then disposed so as to sandwich the pore array sheet 30, whereby a direct current of 1.5 V is applied by a power source 35. mRNAs 36 released have negative charge to make the upper platinum electrode 32 positive. A reference electrode 39 made of silver-silver chloride is provided to apply 0.2 V to the lower platinum electrode 32. Such an operation can guide the mRNAs 36 by electrophoresis into the region 300 for capturing the biomolecules. In addition, to achieve the concentration of the mRNAs by electrophoresis in the lateral direction for further improving the efficiency for capturing the biomolecules, the diameter of the loop of the upper platinum electrode 32 may be 50 μm. In this case, the diameter of the wire is 10 μm.

### (The Description of an Operation Flow)

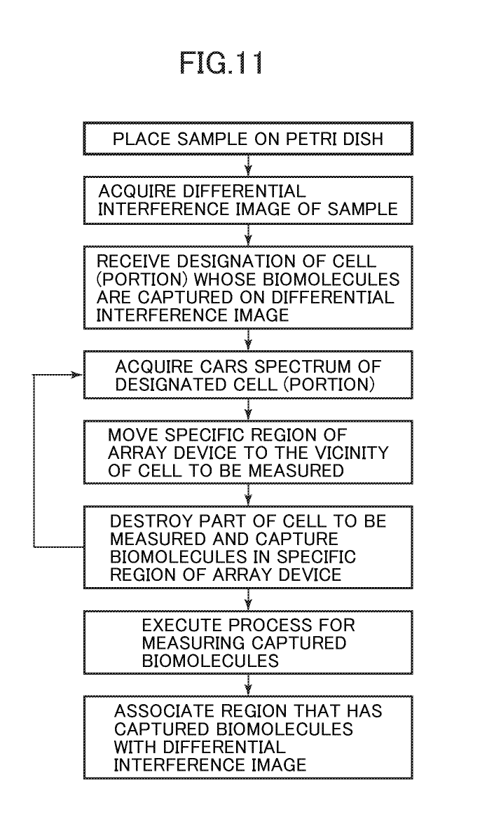

The operation flow of the biomolecule analyzing apparatus according to this embodiment will be described. FIG. 11 illustrates an example of its flowchart.

First, a sample including adherent cultured cells 21, 22, and 23 is placed on a petri dish 20. In this embodiment, cells to be measured are previously cultured by using the petri dish 20, and are then made to adhere onto its bottom face, thereby making the cultured cells. When the sample is a frozen piece, it is placed on the petri dish 20. An alternative sample may include a plurality of cells disposed in a gel in three dimensions. Next, the microscope system is used to acquire the differential interference image of target cells, and the user then decides the target cell for extracting and measuring biomolecules. Then, the computer 11 receives the input of information related to the cell or the cell portion to be measured from the user. Typically, the user often uses a plurality of cells to be measured. In that case, the computer 11 decides the order of the cells for capturing biomolecules, and the XYZ stage 12 is then driven so that the first target cell is located at the center of the visual field. Here, by the method described in the third embodiment, the CARS spectrum of the cell located at the center of the visual field is acquired to store the data in the computer 11.

The computer 11 uses an XYZ stage 34 to bring a specific region of the pore array sheet 30 (e.g., the region 300 at address (1,1)) closer to the vicinity of the cell whose CARS spectrum has been acquired (in the example in FIG. 9, immediately above the cell). Although in this embodiment, the distance between the lower face of the pore array sheet 30 and the petri dish 20 is set to 300 μm, it can be changed according to the type of biomolecules extracted and electrode structure. For instance, the distance is preferably 1 μm to approximately 10 mm. The computer 11 automatically moves the pore array sheet 30 by the XYZ stage 34 according to the previous program. After the computer 11 confirms the completion of the movement, the voltages are applied to the platinum electrodes 32 for electrophoresis. At the same time, to destroy the cell membrane of the cell to be measured, laser light is emitted from the cell destruction laser light source 5 onto the cell. Here, the emission time can be, e.g., 10 seconds, and the electrophoresis driving time can be 60 seconds.

After the destruction of one cell and the capturing of biomolecules in the cell are completed, the computer 11 drives the XYZ stage 12 to locate the registered second target cell at the center of the visual field. After that, the CARS spectrum of the second cell is acquired to store the data in the computer 11. Then, the computer 11 drives the XYZ stage 34 to bring a specific region of the pore array sheet 30 (e.g., the region 300 at address (1,2)) closer to the vicinity of the second target cell (in the configuration example in FIG. 9, immediately above the cell). Laser light is emitted from the cell destruction laser 5 onto the second cell registered in the computer 11. At this time, as described above, the voltages are simultaneously applied to the platinum electrodes 32. Thereafter, the CARS spectra of the designated cells are subsequently acquired to destroy the cells, and biomolecules in the cells are then captured into the specific regions 300 of the pore array sheet 30, thereby executing the process for measuring the captured biomolecules. Finally, the portions of the differential interference image corresponding to the destroyed cells, the regions 300 of the pore array sheet 30 into which the biomolecules have been acquired, and the acquired CARS spectra are associated with each other to show the association results to the user.

One cell is destroyed here, but to acquire data with coarser resolution, mRNAs that are released for electrophoresis at the time of the destruction of a plurality of cells may be captured into one region 300 on the array device. In that case, the plurality of cells may be destroyed simultaneously, or may be subsequently destroyed one by one without moving the array device. In addition, in this embodiment, the acquisition of CARS spectra and the capturing of biomolecules are subsequently carried out with respect to different cells, but for instance, after the acquisition of the differential interference image of the sample, all the CARS spectra of target cells may be measured so that the cells are subsequently destroyed for capturing biomolecules.

By this embodiment, the CARS spectrum and gene expression data of each cell can be acquired. By using this function, the dynamic characteristic of the cell can be confirmed with high precision. To execute such analysis, first, a CARS spectrum is acquired. To confirm the association of the acquired CARS spectrum with the detailed state of a cell selected by the user, the cell is destroyed, and biomolecules in the cell are then captured onto the array device to measure the amount thereof. From the quantification of the biomolecules, the detailed state and type of the cell are identified, thereby associating them with the CARS spectrum, so that the association of the CARS spectrum with the state and type of the cell can be carried out with high precision. The CARS spectrum that can acquire a Raman spectrum can acquire more information related to chemical species to be measured, as compared with a fluorescence confocal microscope that is typically used in single cell analysis, thereby enabling such high precision analysis.

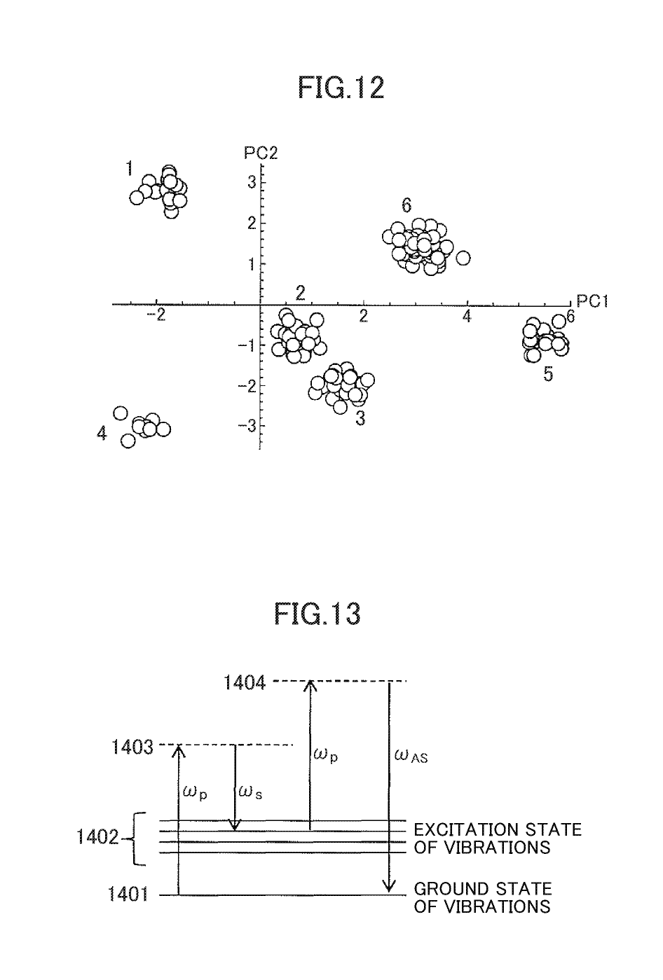

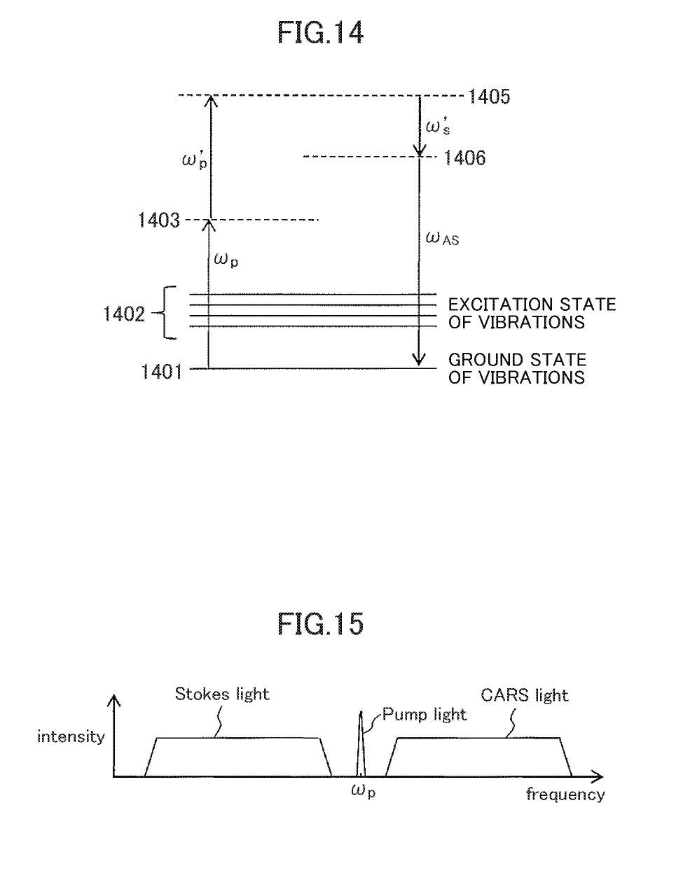

A method for classifying cells by CARS spectra will be described. After the acquisition of CARS spectra, expression analysis of 20 genes in, e.g., 180 cells is carried out to perform principal component analysis. The results are plotted for two higher-order principal components in FIG. 12. PC in the drawing is the abbreviation of a principal component, PC1 denotes a first principal component, and PC2 denotes a second principal component. Each point corresponds to the gene expression data of one cell. In many cases, the points are divided into a plurality of clusters corresponding to the states and types of the cells (in this example, six clusters). Since each point corresponds to one cell in FIG. 12, the association of the cells with the types thereof, even when it cannot be determined only by CARS spectra, is enabled based on the gene expression analysis data. The use of this association allows the computer system to carry out machine learning that determines the association of the acquired CARS spectra with the states and types of the cells, and after the completion of the learning, the states and types of the cells can be classified only by acquiring CARS spectra.

In this example, the principal component analysis is used for clustering based on gene expression in cells, but various methods, such as hierarchical clustering and k-means, are applicable. In addition, as the machine learning, various methods used in data mining, such as a support vector machine, have been known, and any one of them may be used.

In this embodiment, a CARS spectrum is used as a light division spectrum acquired from the sample, but even by using a spontaneous Raman spectrum or a fluorescence spectrum, in place of the CARS spectrum, the same effect can be obtained.

The present invention is not limited to the above embodiments, and includes various modifications. For instance, the above embodiments have been described in detail for clearly understanding the present invention, and do not always include all the above configurations. In addition, part of the configuration of one of the embodiments can be replaced with the configuration of the other embodiments. Further, the configuration of one of the embodiments can be added with the configuration of the other embodiments. Furthermore, part of the configuration of each embodiment can be added with, deleted from, and replaced with the configuration of the other embodiments.

### INDUSTRIAL APPLICABILITY

According to the present invention, the analyzing apparatus that can acquire information at high speed from a large number of samples can be provided, and can accelerate research and development in the field of medicine and pharmaceutical.

### LIST OF REFERENCE SIGNS

2: biomolecule extraction system, 5: cell destruction laser, 11: computer, 21, 22, 23: adherent cultured cell, 30: pore array sheet, 32: platinum electrode, 101: short-pulse laser light source, 104: photonic crystal fiber, 109: objective lens, 110: sample, 113: spectroscope, 114: light division unit, 115: detection unit, 201: CCD camera light reception unit, 401: light, 407: image forming lens, 408: CCD camera

1. An optical analyzing apparatus comprising:
a light source;
a sample holding unit that holds a sample;
an emission optical system that focuses and emits a light flux from the light source onto the sample held by the sample holding unit;
a light division unit that divides light generated from the sample by light emission;
a detection unit that detects the light divided by the light division unit; and
an emission control unit that controls the position of light emission onto the sample by the emission optical system,
wherein the detection unit continues an exposed state over a plurality of positions of light emission onto the sample by the emission control unit, and outputs a spectrum obtained by summing spectra generated from the positions of light emission.

2. The optical analyzing apparatus according to claim 1,
wherein the detection unit outputs a plurality of the summed spectra, and averages the plurality of outputted spectra.

3. The optical analyzing apparatus according to claim 1, further comprising:
an image data acquisition unit that acquires image data of the sample held by the sample holding unit; and
a shape recognition unit that recognizes the shape of the sample based on the acquired image data,
wherein the emission control unit focuses and emits a light flux from the light source onto a specific region of the sample based on the shape of the sample recognized by the shape recognition unit.

4. The optical analyzing apparatus according to claim 1,
wherein the spectrum is a CARS spectrum.

5. The optical analyzing apparatus according to claim 1,
wherein the emission control unit includes a scan mirror,
wherein the scan mirror has a control direction substantially perpendicular to the light division direction of the detection unit.

6. The optical analyzing apparatus according to claim 1,
wherein the emission control unit scans the sample in two dimensions.

7. The optical analyzing apparatus according to claim 1,
wherein the emission control unit scans the sample in three dimensions.

8. A biomolecule analyzing apparatus comprising:
a light source;
a sample holding unit that holds a plurality of cells as a sample;
an observation unit that observes the cells held by the sample holding unit;
an emission optical system that focuses and emits a light flux from the light source onto each cell held by the sample holding unit;
a light division unit that divides light generated from the cell by light emission;
a detection unit that detects the light divided by the light division unit;
an emission control unit that controls the position of light emission onto the cell by the emission optical system;
cell destruction means that destroys the cell held by the sample holding unit; and
a biomolecule capturing device that captures biomolecules in the cell released from the destroyed cell,
wherein the detection unit continues an exposed state over a plurality of positions of light emission onto the cell by the emission control unit, and outputs a spectrum obtained by summing spectra generated from the positions of light emission.

9. The biomolecule analyzing apparatus according to claim 8,
wherein the cell destruction means destroys the cell by laser light emission.
